(19) United States
(12) Patent Application Publication
(10) Pub. No.: US 2008/0247924 A1
(43) Pub. Date: Oct. 9, 2008

(54) POU COLD TANK STERILIZING SYSTEM

(76) Inventor: George Michael Yui, Toronto (CA)

Correspondence Address: MICHAEL RIES, 318 PARKER PLACE, OSWEGO, IL, 60543, US

(21) Appl. No.: 12/062,929

(22) Filed: Apr. 4, 2008

(30) Foreign Application Priority Data
Apr. 4, 2007 (CN) 200720107739.8

(51) Int. Cl. A61L 2/04; F24H 1/18

(52) U.S. Cl. 422/307; 392/441

(57) ABSTRACT

Disclosed is a a POU (point of use) cold tank sterilizing system which is a POU cold tank sterilizing system, mainly used for POU coolers used at home or office. With a cold tank and a hot tank are on the same water level and connected to each other

This application claims priority to Chinese Application 200720107739.8 filed Apr. 4, 2007, the entire disclosure of which is incorporated by reference.

### TECHNICAL FIELD AND BACKGROUND

The present invention relates to a POU (point of use) cold tank sterilizing system. More specifically, the present invention is a POU cold tank sterilizing system, mainly used for POU coolers used at home or office.

With people's quality of life improving, a POU cooler, with its convenient using character is replacing the traditional bottled water cooler. But they both have the same problem after being used for a period of time, they both will unavoidably make water polluted due to bacteria and this is harmful to people. Also it is difficult to wash and make it clean, there are POU coolers with UV or ozone sterilizing in the market, but this kind of sterilizing system is very complicated and difficult to install or uninstall, also ozone has a bad smell and will even make water smell like ozone.

The present invention is a kind of POU cold tank sterilizing system with simple construction, small volume, which will heat the water in the cold tank to sterilize the POU cooler. The present invention is a POU cold tank sterilizing system, having a cold tank and a hot tank, which are connected each other, including an outlet nozzle, on the stated cold tank, installed evaporator, sterilizing heater, thermostat which can be kept from dry burn, inside of cold tank, there is cold thermostat and sterilizing hot thermostat hollow bushing, outside of the cold tank, there is cold tank insulation layer, the stated hot tank bottom, installed hot tank heater and thermostat, outside of the hot tank, there is hot tank insulation layer, on the bottom there is a hot tank bottom cover. The present invention is a POU cooler including the stated cold tank and hot tank are on the same water level and connect to each other, cold tank enter sucker, hot tank enter sucker, cold tank enter solenoid valve and hot tank enter solenoid valve, they are connected each other. The stated sterilizing heater may be installed inside of cold tank. The stated sterilizing heater may be installed outside of cold tank. The stated sterilizing heater may be installed on the bottom of cold tank.

The present invention has the beneficial result of the cold tank and hot tank are on the same water level, which reduce the height and volume of the whole cooler. On the cold tank, there is a sterilizing heater, which can sterilize the cold tank, this avoids having to clean it periodically; two separate solenoid valves will control the inlet water for two separate cold tank and hot tank, they are separate and will not affect each other.

### BRIEF DESCRIPTION OF THE DRAWINGS

The objects, features, and advantages of the present invention will be apparent from the following detailed description of the preferred embodiment of the invention with references to the following drawings.

FIG. 1 is a view of a POU cold tank sterilizing system in accordance with the present invention.

FIG. 2 is a view of a POU cold tank sterilizing system in accordance with the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention, however, the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

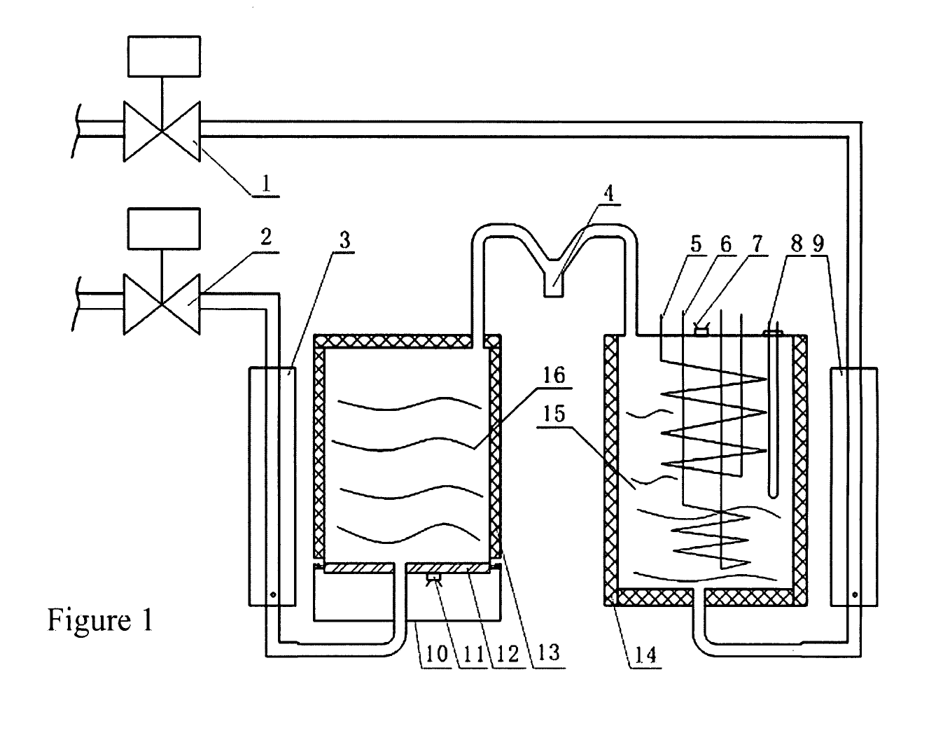

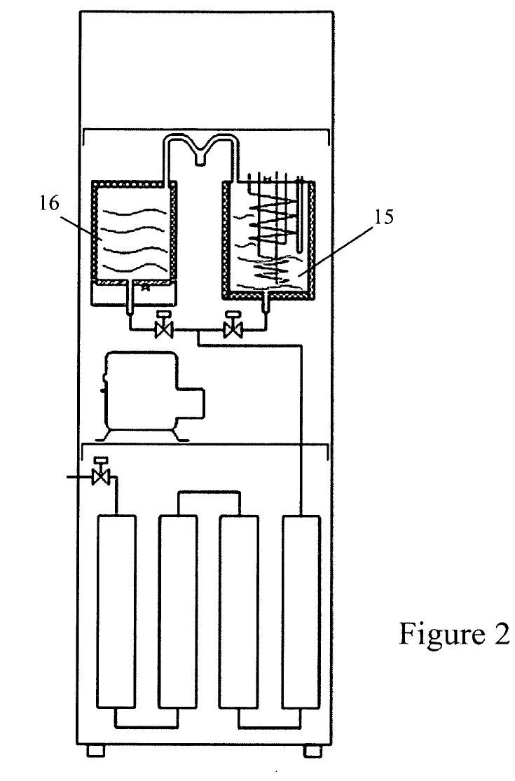

Referring to FIGS. 1 and 2, as in one embodiment is a cold tank 15 and hot tank 16, in the place where connected each other, there is outlet nozzle 4, on the stated cold tank 15, installed evaporator 5, sterilizing heater 6, thermostat 7 which can be kept from a dry burn, in this example, the stated sterilizing heater 6 is put inside of cold tank 15 from the top, at the same time also can be put on the outside or bottom of the cold tank 15, cold thermostat and sterilizing hot thermostat hollow bushing 8 extend into the cold tank 15, there is cold tank insulation layer 14 outside of the cold tank. The stated hot tank bottom, installed hot tank heater 12 and thermostat 11, outside of the hot tank, there is hot tank insulation layer 13, on the bottom there is a hot tank bottom cover 10. The stated cold tank 15 and hot tank 16 are on the same water level and connect to each other and cold tank enter sucker 9, hot tank enter sucker 3, cold tank enter solenoid valve 1 and hot tank enter solenoid valve 2, all are connected to each other.

When the water cooler normally works, cold tank 15 and hot tank 16 are all filled with water. When setting up a sterilizing program, to sterilize the cold tank 15, then the compressor close, i.e. evaporator 5 does not work, at the same time the sterilizing heater 6 starts to work, then heat the water in the cold tank 15, till the water temperature in the cold tank reach the pre set up value (in this example the pre set up value is 88 degrees Celsius), to control the water temperature invariableness through sterilizing and heating thermostat, to keep invariable temperature for 8 minutes; the compressor starts up, evaporator 5 starts to work, at the same time the sterilizing heater 6 stops working.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

What is claimed is:

1. A POU cold tank sterilizing system comprising:
a cold tank and a hot tank connected to each other by an outlet nozzle the cold tank has an installed evaporator, sterilizing heater, cold thermostat which can be kept from a dry burn, inside of the cold tank, there is a cold thermostat and a sterilizing hot thermostat hollow bushing, outside of the cold tank, there is a cold tank insulation layer, a hot tank bottom has installed a hot tank heater and a hot tank thermostat, outside of the hot tank, there is a hot tank insulation layer, on the hot tank bottom there is a hot tank bottom cover, the cold tank and the hot tank are on the same water level and connected to each other, a cold tank enter sucker, a hot tank enter sucker, a cold tank enter solenoid valve and a hot tank enter solenoid valve are connected to each other.

2. The POU cold tank sterilizing system of claim 1 wherein the sterilizing heater is installed inside of the cold tank.

3. The POU cold tank sterilizing system of claim 1 wherein the sterilizing heater is installed outside of the cold tank.

4. The POU cold tank sterilizing system of claim 1 wherein the stated sterilizing heater is installed on a bottom of cold tank.
